(19) United States
(12) Patent Application Publication
(10) Pub. No.: US 2022/0091140 A1
(43) Pub. Date: Mar. 24, 2022

(54) METHOD OF DIAGNOSIS

(71) Applicant: The University of Liverpool, Liverpool (GB)

(72) Inventors: Eithne Costello, Liverpool (GB); William Greenhalf, Liverpool (GB); Lucy Oldfield, Liverpool (GB); Christopher Halloran, Liverpool (GB); Paula Ghaneh, Liverpool (GB)

(73) Assignee: The University of Liverpool, Liverpool (GB)

(21) Appl. No.: 17/431,637

(22) PCT Filed: Feb. 17, 2020
(86) PCT No.: PCT/EP2020/054046

§ 371 Date: Aug. 17, 2021

(30) Foreign Application Priority Data
Feb. 18, 2019 (GB) 1902193.0
Mar. 13, 2019 (GB) 1903424.8

(51) Int. Cl. G01N 33/74; G01N 33/68; G01N 33/577; G01N 33/58

(52) U.S. Cl. CPC G01N 33/74; G01N 33/6869; G01N 2800/067; G01N 33/58; G01N 33/577

(57) ABSTRACT

The present disclosure relates to a diagnostic test for the early detection of pancreatic cancer [i.e. pancreatic ductal adenocarcinoma, ‘PDAC’] or pancreatitis in a cohort of patients selected for an increased risk of PDAC which is associated with diabetes mellitus (DM) and including treatment regimens for the treatment of subjects with PDAC and kits used in the method of the invention.

### FIELD OF THE DISCLOSURE

The present disclosure relates to a diagnostic test for the early detection of pancreatic cancer [i.e. pancreatic ductal adenocarcinoma, ‘PDAC’] or pancreatitis in a cohort of patients selected for an increased risk of developing PDAC which is associated with diabetes mellitus (DM).

### BACKGROUND TO THE DISCLOSURE

The transformation of a normal cell into a cancer cell involves successive somatic and/or heritable mutations in the DNA-sequence of often several genes resulting in functional changes to cellular proteins that confer proliferative advantage and changes in the levels of expression (at the mRNA and protein level) of further genes that are not themselves mutated, but which contribute to the proliferative advantage of cancer cells. Genetic mutations that are inherited in the germ line (from one or both parents) may also predispose to the development of cancer in an individual. However, cancer in general and pancreatic ductal adenocarcinoma (PDAC) in particular, are predominantly not ‘genetically determined’ in the classical sense.

The pancreas has a complex anatomical and cellular composition comprising endocrine cells that release several important hormones into the blood (such as insulin, glucagon and somatostatin) and exocrine cells that secrete lipases and proteases into the intestine to aid digestion. It is the exocrine pancreas that is believed to be the tissue of origin of PDAC. PDAC typically has vague symptoms or is considered asymptomatic until a late stage often allowing the disease to metastasize to other organs before its diagnosis. Untreated metastatic PDAC has a median survival of 3-5 months. Survival for locally advanced disease is 6-10 months. However, the majority of cases are diagnosed in the advanced stages, too late for potentially curative resection. Moreover, chemotherapy is not curative, such that rates of mortality of PDAC approach incidence rates.

In the asymptomatic period leading up to clinical diagnosis the primary tumour sheds potentially metastatic ‘seed’ cells into the circulation. In cases where resection is done when the tumour is <1 cm, five-year survival is dramatically better than if the tumour is larger (75% as compared to about 5%). If all patients could be diagnosed while the tumour was less than 1 cm, survival rates could be dramatically improved by surgical resection. However, there is currently no reliable means to detect asymptomatic PDAC.

The incidence of PDAC is increasing and the projection is that it will surpass breast cancer to become the second leading cause of cancer-related deaths by 2030. As PDAC is typically detected in an advanced stage the majority of PDAC patients are not eligible for potentially curative surgery, however, where surgery is possible, overall survival has shown to be significantly improved.

There are practical barriers to screening the general public for PDAC. Although the disease is a leading cause of cancer deaths, it is relatively rare and occurs only at an incidence of 0.014%. Thus, a screening test would have to be extremely specific and nearly approaching 100% in order to avoid a large number of false positives. However, no such screening tests currently exist. CA19-9 is the only biomarker in routine clinical use for the management of PDAC. However, due to its low sensitivity and specificity the false positive rate in screening tests is exceptionally high, and the additional tests to confirm the diagnosis are expensive, making the screen inefficient and costly.

Other markers are known in the art. U.S. Pat. No. 9,863,960 discloses a method for diagnosing pancreatic cancer by measuring the presence of Interleukin-1 receptor antagonist (IL-1Ra). WO2017109518 discloses methods of determining the protein glycosylation signature of target proteins to determine if a subject has pancreatic cancer. Hyper-glycosylation of CA-19-9 and CEA was shown to be associated with pancreatic cancer. Further, US20170003294 and WO2015157557 disclose diagnostic tests for the detection of diseases such as pancreatic cancer or pancreatic inflammatory conditions comprising determining the presence or absence of a numerous range of biomarkers.

Research has shown that at the time of PDAC diagnosis, the majority of PDAC patients have diabetes mellitus (DM)^{1, 2}. By contrast, the prevalence of DM in individuals with lung, breast, prostate, and colorectal cancers is no higher than non-cancer controls³. The relationship between PDAC and DM is complex. Long-standing DM increases the risk of PDAC, but only by approximately twofold (roughly equivalent to smoking). However, epidemiological data indicate that PDAC can cause DM, with new-onset DM an early warning sign of the presence of PDAC⁴. In approximately 50% of PDAC cases diabetes is of recent onset 3 years), making individuals with new-onset DM the largest high-risk group for PDAC. The average time between the diagnosis of new-onset DM and the subsequent diagnosis of PDAC is 13 months⁵.

While this represents a substantial opportunity for earlier detection of PDAC, there are challenges. The incidence of DM in the general population is rising, with an estimated 200,000 new cases of type 2 DM (T2DM) diagnosed each year in the UK. In 10% of new-onset DM cases, DM is secondary to pancreatic disease (PDAC, chronic pancreatitis and other) and is known as type 3cDM; however, in most cases it is misdiagnosed as T2DM.

This disclosure relates to the characterisation of a highly specific test for the detection of PDAC or pancreatitis in an individual with new-onset diabetes which measures the levels of adiponectin and IL-1Ra in a biological sample to distinguish diabetes of the exocrine pancreas (Type 3c, including PDAC and pancreatitis-associated DM) from T2DM, allowing the former to enter screening for PDAC.

### STATEMENT OF THE INVENTION

According to an aspect of the invention there is provided an immunoassay to determine whether a subject has elevated levels of an adiponectin polypeptide and an IL-1Ra polypeptide comprising the steps:
i) obtaining a biological sample from a subject to be tested;
ii) forming a preparation comprising said sample and an antibody or antibodies that bind adiponectin and an antibody or antibodies that bind IL-1Ra to form an antibody/adiponectin polypeptide complex and an antibody/IL-1Ra polypeptide complex;
iii) detecting each complex; and
iv) comparing the level of adiponectin and IL-1Ra to a relevant matched control.

The disclosure compares samples obtained from a subject to be tested with a relevant matched control. For example, a relevant matched control could be a subject that has or is suspected of having new-onset DM without associated pancreatic cancer.

According to an aspect of the invention there is provided an immunoassay to determine whether a subject is suspected of having early stage pancreatic ductal adenocarcinoma or pancreatitis comprising the steps:
i) obtaining a biological sample from a subject to be tested;
ii) forming a preparation comprising said sample and an antibody or antibodies that bind an adiponectin and an antibody or antibodies that bind IL-1Ra to form an antibody/adiponectin polypeptide complex and an antibody/IL-1Ra polypeptide complex;
iii) detecting each complex; and
iv) comparing the level of adiponectin polypeptide and IL-1Ra polypeptide to a relevant matched control.

Pancreatitis is the inflammation of the pancreas which can be acute or chronic. In acute pancreatitis the pancreas becomes inflamed for a short period of time often without lasting damage; in chronic pancreatitis the pancreas is permanently damaged and is unable to produce any or enough amounts of digestive fluids. The symptoms of chronic pancreatitis are often very similar to pancreatic cancer.

In a preferred method of the invention said pancreatitis is acute or chronic pancreatitis, preferably chronic pancreatitis.

In a preferred method of the invention the adiponectin polypeptide comprises the amino acid sequence set forth in SEQ ID NO: 1, or a polymorphic sequence variant thereof.

In a preferred method of the invention the level of the adiponectin polypeptide is increased at least 2-fold compared to said normal matched control.

In a preferred method of the invention the IL-1Ra polypeptide comprises the amino acid sequence set forth in SEQ ID NO: 2, or a polymorphic sequence variant thereof.

In a preferred method of the invention the level of the IL-1Ra polypeptide is increased at least 2-fold compared to said normal matched control.

In a preferred method of the invention said subject is pre-screened to determine whether the subject is pre-diabetic or has early stage T2DM.

Symptoms of diabetes mellitus can vary but include excessive thirst, frequent urination, tiredness, weight loss and loss of muscle bulk, frequent episodes of thrush, cuts or wounds that heal slowly and blurred vision. Diabetes mellitus is typically diagnosed by measuring blood sugar in the blood.

In a preferred method of the invention said subject is tested for diabetes mellitus

In a preferred method of the invention the level of CA19-9 is determined as a measure of whether the subject has or is predisposed to PDAC.

In an alternative method of the invention the level of CA19-9 is determined as a measure of whether the subject has or is predisposed to pancreatitis.

In a preferred method of the invention said pancreatitis is acute or chronic pancreatitis, preferably chronic pancreatitis.

Many monoclonal antibodies have been developed against the CA19-9 antigen, which is a carbohydrate structure called sialyl-Lewis A (part of the Lewis family of blood group antigens) with the sequence Neu5Acα2,3Galβ1,3(Fucα1,4) GlcNAc. Sialyl Lewis A is synthesized by glycosyltransferases that sequentially link the monosaccharide precursors onto both N-linked and O-linked glycans.

In a preferred method of the invention the level of CA19-9 is determined by an immunoassay.

In a preferred method of the invention said immunoassay is an ELISA or bead-based immunoassay.

Alternative formats for the disclosed immunoassay are available, for example, sandwich assay, western blot, mass spectrometry-based detection, an ELISA or bead-based immunoassay, for example Luminex®. However, the working of the invention is not limited to a particular immunoassay format and other formats available to the skilled person are available.

In a preferred method of the invention said biological sample is selected from the group consisting of: urine, blood, blood plasma or serum.

Urine is an important source of biomarkers and can be collected continuously, in a non-invasive fashion and is typically preferred over blood sampling. IL-1Ra detection in urine is a standard procedure (21) and commercially available kits for the detection of IL-1Ra in urine are widely available (Table 1). Adiponectin can be detected in urine from healthy subjects with commercially available kits such as Luminex assay kit.

In a preferred method of the invention said biological sample is urine.

In a preferred method of the invention said antibody is polyclonal serum.

In an alternative method of the invention said antibody is a monoclonal antibody.

In a preferred method of the invention the subject is imaged, for example by tomography or magnetic resonance imaging, to determine whether the subject has early stage pancreatic ductal adenocarcinoma or pancreatitis.

According to a further aspect of the invention there is provided a treatment regimen for early stage pancreatic ductal adenocarcinoma or pancreatitis comprising:
i) conducting the method according to the invention on a subject suspected of having early stage pancreatic ductal adenocarcinoma or pancreatitis; and
ii) treating the subject for early stage pancreatic ductal adenocarcinoma or pancreatitis if the method determines said subject has or is susceptible to early stage pancreatic ductal adenocarcinoma or pancreatitis.

In a preferred embodiment of the invention said treatment is the resection of tumour tissue.

In an alternative embodiment of the invention said treatment is the administration of one or more chemotherapeutic agents.

In a preferred embodiment of the invention said therapeutic agent is selected from the group consisting of FOLFIRINOX (oxaliplatin, leucovirin, irinotecan and fluorouracil), Gemcitabine, GemCap (gemcitabine and capecitabine), FOLFOX (oxaliplatin, fluorouracil and folinic acid) and Nab-paclitaxel with gemcitabine.

According to a further aspect of the invention there is provided a solid support comprising immobilised antibodies that specifically bind adiponectin and/or IL-1Ra.

In a preferred embodiment of the invention said solid support further comprises an immobilised antibody that specifically binds CA19-9.

Preferably, antibodies that specifically bind adiponectin, IL-1Ra and CA19-9 are monoclonal antibodies.

According to a further aspect of the invention there is provided a point of care device comprising a solid support according to the invention.

According to a further aspect of the invention there is provided a kit comprising or consisting of:
an antibody that specifically binds an adiponectin polypeptide;
an antibody that specifically binds an IL-1Ra polypeptide; and

secondary antibodies that bind said monoclonal antibody that binds an adiponectin polypeptide and a secondary antibody that binds an IL-1Ra polypeptide wherein said secondary antibodies comprise separate detectable labels.

In a preferred embodiment of the invention there is provided an antibody that binds CA-19 and a secondary antibody that binds CA19-9 comprising a separately detectable label.

Preferably, said antibodies that specifically bind adiponectin, IL-1Ra and CA19-9 are monoclonal antibodies.

According to a further aspect of the invention there is provided a method to determine whether a subject should be imaged for early stage pancreatic ductal adenocarcinoma or pancreatitis comprising or consisting of the steps:
i) obtaining a biological sample from a subject to be tested;
ii) forming a preparation comprising said sample and an antibody or antibodies that bind an adiponectin and an antibody or antibodies that bind IL-1Ra to form an antibody/adiponectin polypeptide complex and an antibody/IL-1Ra polypeptide complex;
iii) detecting each complex;
iv) comparing the level of adiponectin polypeptide and IL-1Ra polypeptide to a relevant matched control; and
v) determining whether the subject should be imaged to determine whether the subject has early stage pancreatic ductal adenocarcinoma or pancreatitis.

In a preferred method of the invention the subject is imaged, for example by tomography or magnetic resonance imaging, to determine whether the subject has early stage pancreatic ductal adenocarcinoma or pancreatitis.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

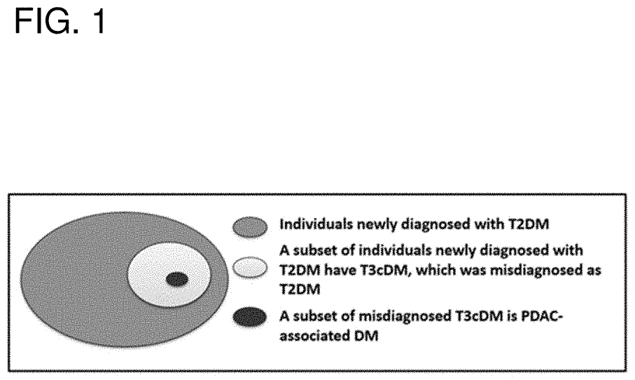

FIG. 1: ˜10% of all individuals who are newly diagnosed with T2DM actually have T3cDM. PDAC-associated DM accounts for 8 to 10% of T3cDM. T3cDM, type 3c diabetes mellitus; T2DM, type 2 diabetes mellitus;

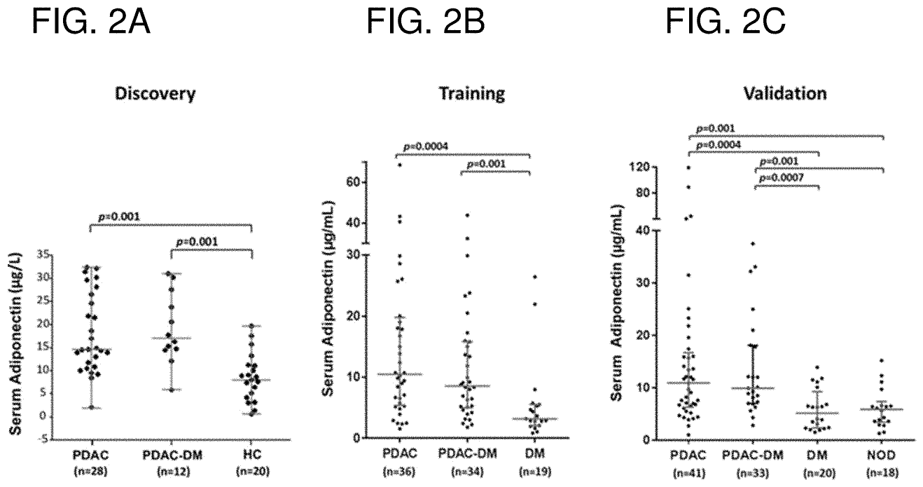

FIG. 2: Serum adiponectin measured in three independent cohorts shows that levels of this adipokine are higher in PDAC-DM patients compared to DM controls and can distinguish PDAC from long-standing and new-onset T2DM. PC, Pancreatic cancer; PDAC-DM, pancreatic cancer-associated diabetes; DM, longstanding diabetes; NOD, new-onset diabetes; HC, healthy controls;

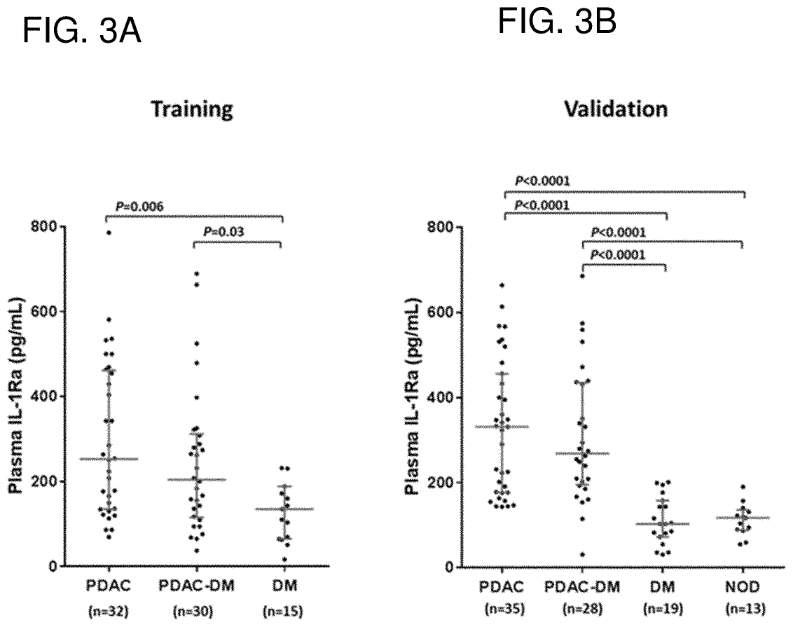

FIG. 3: Circulating levels of IL-1Ra measured in two independent cohorts. IL-1Ra is up regulated in patients with PDAC and PDAC-DM compared to those with T2DM, regardless of duration (A, B). PDAC, Pancreatic cancer; PDAC-DM, pancreatic cancer-associated diabetes; DM, long-standing diabetes; NOD, new-onset diabetes.

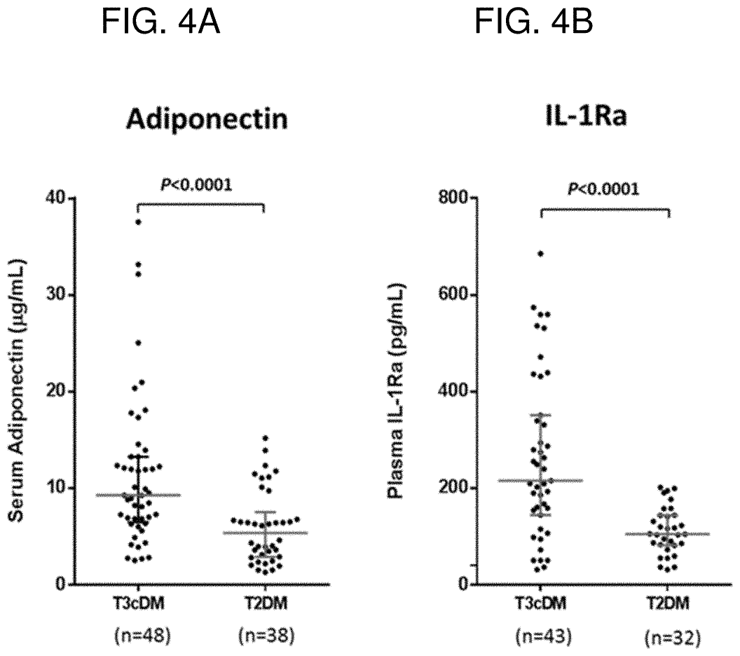

FIG. 4: Serum levels of adiponectin (A) and plasma levels of IL-1Ra (B) are significantly elevated in patients with T3cDM (PDAC- and chronic pancreatitis-associated) compared to those with T2DM. T3cDM, type 3c diabetes mellitus; T2DM, type 2 diabetes mellitus.

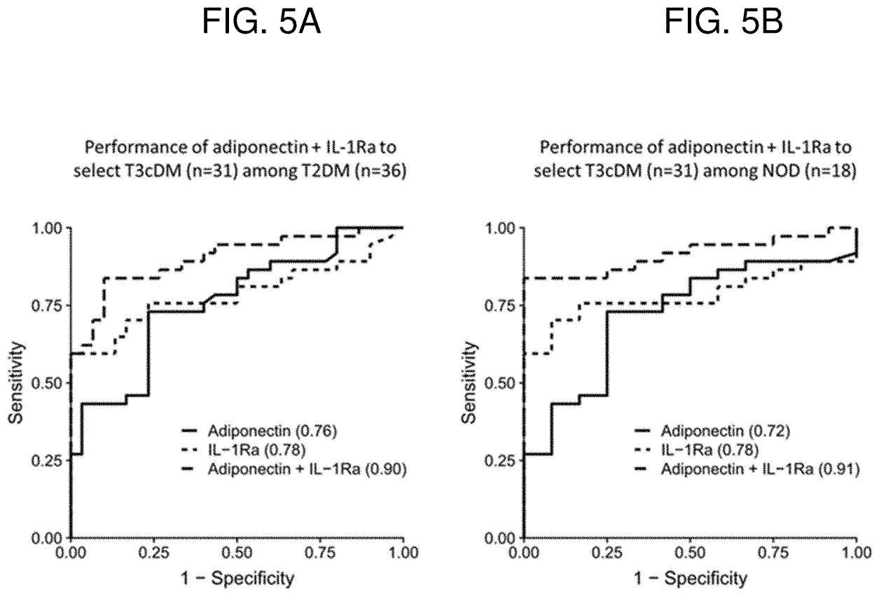

FIG. 5: In combination, serum adiponectin and plasma IL-1Ra showed good performance in the discrimination of type 3c diabetes from type 2 diabetes, regardless of diabetes duration (A) or compared to new-onset alone (B) (T3cDM, type 3c diabetes mellitus; T2DM, type 2 diabetes mellitus; NOD, new-onset diabetes mellitus).

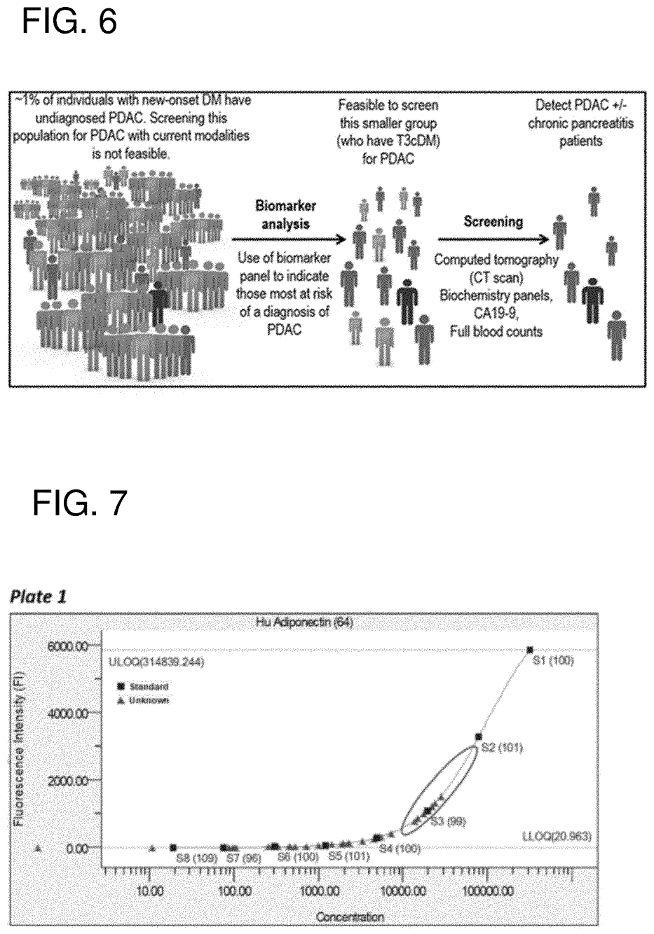

FIG. 6: Biomarker-mediated identification of PDAC (black individual) amongst new-onset DM individuals (all colours) and pathway to diagnosis. Distinguishing T3cDM (dark grey and black individuals) from T2DM (grey individuals) will identify a PDAC-enriched population, making screening of this subpopulation feasible. All T3cDM patients require management that differs from that typically offered to T2DM patient. Our focus is on the detection of PDAC. PDAC accounts for 10% of cases in the T3cDM group.

FIG. 7: Calibration curve obtained from Plate 1 of Luminex analysis displaying the measured adiponectin concentration (pg/mL) in each standard and sample against fluorescence intensity. Optimal dilutions of urine are placed on the linear section of the curve between S2 and S4 (circled in red).

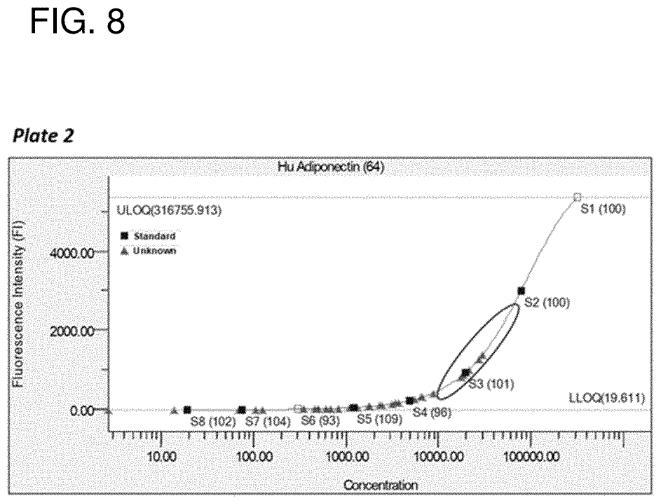

FIG. 8: Calibration curve obtained from Plate 2 of Luminex analysis displaying the measured adiponectin concentration (pg/mL) in each standard and sample against fluorescence intensity. Optimal dilutions of urine are placed on the linear section of the curve between S2 and S4 (circled in red).

### MATERIALS AND METHODS

Patient Groups

Serum and plasma samples from individuals with pancreatic cancer (pre-surgical) and healthy subjects were obtained from the University of Liverpool GCP Laboratory Facility Biobank. Serum and plasma samples from individuals with diabetes were collected at the Royal Liverpool University Hospital after referral from diabetes clinics and primary care centres. All participants gave written informed consent using approved ethics protocols, at the Royal Liverpool University Hospital (Ethics Identifier: 11/NW/0083 and 16/LO/1630).

Sample Cohorts

Three independent sample sets were analysed. The first discovery set (n=60) consisted of 40 individuals with histologically confirmed PDAC and 20 healthy control individuals. Individuals with PDAC were further subcategorised into those with a positive diagnosis of diabetes (12 patients, HbA1c ≥48 mmol/mol) and those with negative or no defined diabetes diagnosis (28 patients, HbA1c <47 mmol/mol or no data). To determine levels of candidate markers in individuals with type 2 diabetes mellitus, a second independent training sample set (n=135) was analysed. This included 80 individuals with histologically confirmed PDAC (40 with diagnosed diabetes and 40 with a negative diabetes diagnosis), 20 individuals with chronic pancreatitis (10 with diagnosed diabetes and 10 with a negative diabetes diagnosis), 20 individuals with long-standing (>3 years post-diagnosis) type 2 diabetes mellitus and 15 healthy subjects. A third independent validation set (n=175) was used to evaluate the levels of candidate markers in individuals with new-onset type 2 diabetes mellitus (<3 years post diagnosis). The validation set consisted of 78 individuals with histologically confirmed PDAC (37 with diagnosed diabetes and 41 with a negative diabetes diagnosis), 39 individuals with chronic pancreatitis (19 with diagnosed diabetes and 20 with a negative diabetes diagnosis), 20 individuals with long-standing (>3 years post-diagnosis) type 2 diabetes mellitus, 18 individuals with new-onset (3 years post-diagnosis) type 2 diabetes mellitus and 20 healthy subjects.

Individuals with PDAC had resectable disease and surgery was undertaken with curative intent.

Sample Collection

Blood was collected in Sarstedt Monovette Serum Z tubes or K+EDTA tubes (Sarstedt Ltd, Leicester, UK) and allowed to stand for 30 minutes at room temperature before centrifugation at 800×g for 10 min for serum fractionation and 16000×g for 1 minute for plasma fractionation. The serum and plasma fractions were aliquoted into cryotubes and stored at −80° C. prior to use.

Discovery Analysis

Luminex and mass spectrometry-based isobaric tags for relative and absolute quantification (iTRAQ) were performed using a discovery subset of samples; PDAC (n=40) and healthy controls (n=20; total n=60) as described previously^{6, 7}. A list of significantly altered proteins generated from our iTRAQ data was uploaded into Ingenuity Pathway Analysis (IPA) software (http://www.ingenuity.com). Both a Core Analysis and Biomarker Filter were performed to identify those proteins associated with metabolic disease pathways and diabetes.

Training Analysis

Candidate biomarkers generated through IPA were assessed using immunoassays on 135 serum and plasma samples; PDAC (n=40 with diabetes, n=40 without diabetes), chronic pancreatitis (n=10 with diabetes, n=10 without diabetes), long-standing type 2 diabetes (n=20), and healthy controls (n=20).

Validation Analysis

The most promising markers emerging from training analysis were assessed using immunoassays on 175 serum and plasma samples; PDAC (n=37 with diabetes, n=41 without diabetes), chronic pancreatitis (n=19 with diabetes, n=20 without diabetes), long-standing type 2 diabetes (n=20), new-onset type 2 diabetes (n=18) and healthy controls (n=20).

Biomarker Measurement and Data Filtering

Serum adiponectin and plasma IL-1Ra levels were measured using commercially available Luminex (Bio-Plex Pro Diabetes Adiponectin Assay and Bio-Plex Pro Human Cytokine 27-Plex Assay, respectively; Bio-Rad, UK) on a Bio-Plex 200 System (Bio-Rad, UK). All samples were measured in duplicate following the manufacturers' instructions with inter-plate variability assessed using 3 quality controls per plate.

Biomarker concentrations were determined from standard curves of positive control proteins using four- or five-parameter logistic regression models. Inter-plate variation of less than or equal to 15% was considered acceptable; any plate falling outside of this was repeated. Quantified biomarkers with concentrations falling outside of the linear range and those with duplicate measurements having a coefficient of variance (CV) >20%, were removed from the dataset.

Blood glucose (HbA1c mmol/mol) was measured by the Royal Liverpool University Hospital Clinical Biochemistry Department, using an International Federation of Clinical Chemistry-approved method.

Statistical Analysis

JMP Version 14 (SAS Institute Inc., Cary, N.C., USA) and RStudio Version 1.1.463 (Integrated Development for R, RStudio Inc., Boston, Mass., USA; http://rstudio.com/), were used. Protein expression data was analysed using a two-tailed Mann-Whitney U test and a stepwise regression model was used to select the most promising marker combinations. The diagnostic accuracy of each candidate marker (adiponectin and IL-1Ra), both alone and in combination, was assessed by ROC analyses.

Urine Sample Collection

Urine samples were obtained from two healthy subjects and processed with—and without the inclusion of protease inhibitors. Urine samples were subjected to the following dilutions: (1:1), (1:2), (1:4), (1:5), (1:10), (1:20), (1:100). Plasma controls were obtained from one healthy subject and one pancreatic cancer patient and were prepared to a (1:400) dilution. Adiponectin levels were measured using the Bio-Plex Pro Human Diabetes Adiponectin Assay (Bio-Rad, #171A7003M).

Data was processed using Bio-Plex Manager Software. Adiponectin concentrations were averaged and calculated Coefficient of variation (CV) values <15% were considered acceptable for standards and samples. Data points with CV >15% were marked as outliers, consequently generating an 8-point calibration curve for further analysis.

A quality control check was also carried out using the adiponectin concentrations of the plasma controls where CV values <15% were considered acceptable.

TABLE 1
commercially available kits for the detection of IL-1Ra in urine

| NAME/Vendor | SAMPLE TYPE | CITATION |
|---|---|---|
| Human IL-1ra ELISA Kit | EDTA Plasma, Urine, | (22) |
| (ABCAM, ab211650) | Serum, Saliva, cell culture | |
| Human IL-1ra ELISA Kit, | Urine, plasma, serum, cell | (23) |
| (SIGMA ALDRICH, RAB0283) | culture | |
| Human IL-1ra ELISA Kit, | Urine, plasma | Not |
| Fluorescent (ABCAM, | | referenced |
| ab229435) | | |
| IL1RA Human ELISA Kit | Urine, Plasma, Serum, | (24) |
| (THERMOFISHER, | Supernatant | |
| BMS2080) | | |

### Example 1

Distinguishing New-Onset DM Caused by PDAC from T2DM would Allow for Earlier Diagnosis of PDAC.

The incidence of DM in the general population is rising, with an estimated 200,000 new cases of type 2 DM (T2DM) diagnosed each year in the UK. In 10% of new-onset DM cases, DM is secondary to pancreatic disease (PDAC, chronic pancreatitis and other) and is known as type 3c DM (T3cDM); however, in the majority of cases it is misdiagnosed as T2DM (FIG. 1)⁸. PDAC-associated DM accounts for 8-10% of cases of misdiagnosed new-onset T3cDM (equivalent of 0.8-1% of new diagnoses of T2DM).

Identifying the 0.8-1% of individuals with new-onset diabetes who have underlying PDAC-associated DM is not feasible using current screening modalities. Distinguishing T3cDM from T2DM would identify a PDAC-enriched population, making screening of this subpopulation feasible. Screening would be facilitated if diagnostic biomarkers were established that would, in combination with other clinical features, aid in the identification of these high-risk individuals.

To improve early diagnosis of PDAC we have extensively analysed serum and plasma for the detection and development of protein biomarkers^{6,7,9-15}. Using isobaric Tags for Relative and Absolute Quantification (iTRAQ) coupled with liquid chromatography-tandem mass spectrometry (LC-MS/MS) and immunoassays (Luminex, Rules Based Medicine, ELISA and western blotting) we have carried out comprehensive discovery programs.

Using >500 diagnostic and control samples from Liverpool our discovery work has identified 379 differentially regulated serum proteins in PDAC. Ingenuity Pathway Analysis (IPA) selected those differentially expressed proteins with an association to metabolic disease pathways and diabetes. Analysis of a subset of these samples, where DM status was known, enabled us to observe differences between pancreatic cancer (regardless of DM status) and healthy controls. Subsequent analysis of 19 candidate biomarkers using independent serum and plasma training and validation sets highlighted adiponectin and interleukin-1 receptor antagonist (IL-1Ra) as important potential components of a novel panel for detection of T3cDM including PDAC-DM.

Low circulating adiponectin (<4 μg/mL) is associated with T2DM¹⁶. We, however, observed significantly elevated serum levels of this adipokine in PDAC patients, regardless of DM status, versus healthy controls in independent discovery, training and validation sample sets (FIG. 2). In our training set, for PDAC and DM individuals (n=89), adiponectin was shown to be elevated in both diabetic and non-diabetic PDAC patients compared with long-standing DM controls (p=0.001 and p=0.0004, respectively; FIG. 2B). Adiponectin levels were similar between chronic pancreatitis and PDAC patients and were unaffected by jaundice (data not shown), an important consideration as we have previously demonstrated the influence of elevated bilirubin on the measured levels of certain proteins in blood¹².

Validation in an independent cohort (n=175) confirmed that significantly elevated levels of adiponectin are present in individuals with PDAC compared to those with T2DM. Elevation in serum adiponectin was observed regardless of the duration of DM (both long-standing and new-onset T2DM; FIG. 2C). Adiponectin was also found to be elevated in diabetic (n=19) and non-diabetic (n=20) chronic pancreatitis (CP) patients compared with T2DM controls (n=39, data not shown).

In individuals with newly diagnosed DM, who have a notable reduction in adiponectin level at the time of diagnosis¹⁷ higher levels of adiponectin could highlight the presence of PDAC. Furthermore, lower circulating levels of adiponectin are a predictor for T2DM and it is increasingly suggested that adiponectin measurement would provide a means of earlier identification of impending diabetes¹⁸. With increased awareness of the role of adiponectin in DM diagnosis, an opportunity to detect PDAC-associated DM arises.

Our data suggest that individuals presenting with clinical indicators of DM, but with normal to high adiponectin, should be tested for pancreatic cancer.

Interleukin-1 receptor antagonist (IL-1Ra) reduces the endogenous activity of the IL-1 family of pro-inflammatory cytokines, protects β-cells from the destructive effects of high glucose exposure, and while the β-cell expression of IL-1Ra is reduced in patients with T2DM¹⁹, blood levels are increased. We found IL-1Ra expression was unaffected by jaundice and elevated in PDAC compared to healthy controls in both serum and plasma in a Luminex based discovery program (p<0.05, data not shown²⁰). This observation was confirmed in plasma in independent training and validation sets, with levels shown to be significantly elevated in both PDAC and PDAC-DM compared to T2DM (p<0.006 and <0.03 respectively; FIGS. 3A and B) and new-onset T2DM (p<0.0001; FIG. 3B). Our data support the use of circulating IL-1Ra as a valuable marker for earlier detection of PDAC in high-risk individuals newly diagnosed with DM.

While blood levels of adiponectin and IL-1Ra were found to be elevated in PDAC and also CP (not shown) irrespective of DM status, their potential role as markers for the identification of T3cDM (PDAC- and CP-associated) among individuals diagnosed with T2DM was highlighted, with a significant separation in median biomarker levels observed between the two groups (p<0.0001; FIGS. 4A and B).

The increase in expression of both adiponectin and IL-1Ra in PDAC and chronic pancreatitis (CP), irrespective of DM status, compared with T2DM controls, indicates a type 3c specific effect (FIG. 4). Moreover, the combination of adiponectin with IL-Ra demonstrates good performance in distinguishing of T3cDM (PDAC- and CP-associated) from T2DM (AUC 0.90, FIG. 5A), with sensitivity/specificity of 73.7%/83.7%. More specifically, in the distinction of T3cDM from among individuals with new-onset T2DM, the combination of adiponectin and IL-1Ra achieved an AUC of 0.91 with sensitivity and specificity of 83.7% (FIG. 5B).

Our test is targeted at the estimated 200,000 new individuals diagnosed with T2DM each year in the UK. Our markers enable us to distinguish new-onset DM caused by common T2DM from new-onset DM caused by T3cDM. Identifying individuals with T3cDM (comprising both PDAC and CP) will select a subpopulation greatly enriched for PDAC (FIG. 6). In turn, identifying those with the highest risk of a PDAC diagnosis will make future screening (of this subpopulation) using existing modalities (EUS, CT/MRI scan, biochemistry panels) feasible (FIG. 6). Our test will also select out patients with CP.

### Example 2

Adiponectin was successfully detected in urine using a Luminex assay (Table 2), with concentrations measurable in line with generated calibration curves (FIG. 7 and FIG. 8). Optimal dilutions of urine were (1:1) and (1:2) as these samples lay between the linear section of the standard curve. Little variance in adiponectin concentrations were observed for urine processed with—and without protease inhibitors. Adiponectin levels are measurable in urine. Urine is thus suitable for the measurement of adiponectin and IL-1RA.

TABLE 2
Table displaying average adiponectin concentration in patient
urine samples with and without protease
inhibitors and their corresponding CV values.

| | | Average | Average | |
|---|---|---|---|---|
| | | [Adiponectin] | [Adiponectin] | |
| | | (ng/ml) | (ng/ml) | |
| | Healthy | With Protease | Without Protease | |
| Dilution | Subject | Inhibitors | Inhibitors | % CV |
| (1:1) | Subject A | 18.38 | 16.36 | 8.21 |
| | Subject B | CV > 15% | 25.91 | — |
| (1:2) | Subject A | 11.10 | 10.51 | 3.85 |
| | Subject B | 12.19 | 16.06 | 19.35 |
| (1:4) | Subject A | 6.48 | 8.30 | 17.40 |
| | Subject B | 11.48 | 14.19 | 14.89 |
| (1:5) | Subject A | 6.35 | 8.15 | 17.53 |
| | Subject B | CV > 15% | 13.54 | — |
| (1:10) | Subject A | 5.74 | 7.71 | 20.66 |
| | Subject B | 11.01 | 10.52 | 3.22 |
| (1:20) | Subject A | 8.04 | 6.09 | 19.45 |
| | Subject B | 11.52 | 9.53 | 13.40 |
| (1:100) | Subject A | 9.80 | 7.41 | 19.62 |
| | Subject B | CV > 15% | 10.74 | — |

### REFERENCES

1. Permert J, Ihse I, Jorfeldt L, et al. Pancreatic cancer is associated with impaired glucose metabolism. Eur J Surg 1993; 159:101-7.
2. Pannala R, Leirness J B, Bamlet W R, et al. Prevalence and clinical profile of pancreatic cancer-associated diabetes mellitus. Gastroenterology 2008; 134:981-7.
3. Aggarwal G, Kamada P, Chari S T. Prevalence of diabetes mellitus in pancreatic cancer compared to common cancers. Pancreas 2013; 42:198-201.
4. Andersen D K, Korc M, Petersen G M, et al. Diabetes, Pancreatogenic Diabetes, and Pancreatic Cancer. Diabetes 2017; 66:1103-1110.
5. Sah R P, Nagpal S J, Mukhopadhyay D, et al. New insights into pancreatic cancer-induced paraneoplastic diabetes. Nat Rev Gastroenterol Hepatol 2013; 10:423-33.
6. Jenkinson C, Elliott V L, Evans A, et al. Decreased Serum Thrombospondin-1 Levels in Pancreatic Cancer Patients Up to 24 Months Prior to Clinical Diagnosis: Association with Diabetes Mellitus. Clin Cancer Res 2016; 22:1734-43.
7. Shaw V E, Lane B, Jenkinson C, et al. Serum cytokine biomarker panels for discriminating pancreatic cancer from benign pancreatic disease. Mol Cancer 2014; 13:114.
8. Woodmansey C, McGovern A P, McCullough K A, et al. Incidence, Demographics, and Clinical Characteristics of Diabetes of the Exocrine Pancreas (Type 3c): A Retrospective Cohort Study. Diabetes Care 2017; 40:1486-1493.
9. Jenkinson C, Elliott V, Menon U, et al. Evaluation in pre-diagnosis samples discounts ICAM-1 and TIMP-1 as biomarkers for earlier diagnosis of pancreatic cancer. J Proteomics 2014; 113C:400-402.
10. O'Brien D P, Sandanayake N S, Jenkinson C, et al. Serum CA19-9 is significantly upregulated up to 2 years before diagnosis with pancreatic cancer: implications for early disease detection. Clin Cancer Res 2015; 21:622-31.
11. Sinclair J, Timms J F. Quantitative profiling of serum samples using TMT protein labelling, fractionation and LC-MS/MS. Methods 2011; 54:361-9.
12. Tonack S, Jenkinson C, Cox T, et al. iTRAQ reveals candidate pancreatic cancer serum biomarkers: influence of obstructive jaundice on their performance. Br J Cancer 2013; 108:1846-53.
13. Tonack S, Neoptolemos J P, Costello E. Analysis of serum proteins by LC-MS/MS. Methods Mol Biol 2010; 658:281-91.
14. Tonack S, Aspinall-O'Dea M, Jenkins R E, et al. A technically detailed and pragmatic protocol for quantitative serum proteomics using iTRAQ. J Proteomics 2009; 73:352-6.
15. Yan L, Tonack S, Smith R, et al. Confounding effect of obstructive jaundice in the interpretation of proteomic plasma profiling data for pancreatic cancer. J Proteome Res 2009; 8:142-8.
16. Kawada T, Hasegawa M. Predictive ability of serum high-molecular-weight adiponectin in combination with serum insulin and serum C-reactive protein for the presence of metabolic syndrome. Ann Hum Biol 2012; 39:108-12.
17. Herder C, Carstensen M, Ouwens D M. Anti-inflammatory cytokines and risk of type 2 diabetes. Diabetes Obesity & Metabolism 2013; 15:39-50.
18. Anderson S G, Dunn W B, Banerjee M, et al. Evidence That Multiple Defects in Lipid Regulation Occur before Hyperglycemia during the Prodrome of Type-2 Diabetes. Plos One 2014; 9.
19. Marculescu R, Endler G, Schillinger M, et al. Interleukin-1 receptor antagonist genotype is associated with coronary atherosclerosis in patients with type 2 diabetes. Diabetes 2002; 51:3582-5.
20. Shaw V E, Lane B, Jenkinson C, et al. Serum cytokine biomarker panels for discriminating pancreatic cancer from benign pancreatic disease. Molecular Cancer 2014; 13.
21. Zwiech R. Predictive value of conjointly examined IL-1ra, TNF-R I, TNF-R II, and RANTES in patients with primary glomerulonephritis. J Korean Med Sci. 2013; 28(2):261-7.
22. Karpenko M N, Vasilishina A A, Gromova E A, Muruzheva Z M, Miliukhina I V, Bernadotte A. Interleukin-1beta, interleukin-1 receptor antagonist, interleukin-6, interleukin-10, and tumor necrosis factor-alpha levels in CSF and serum in relation to the clinical diversity of Parkinson's disease. Cell Immunol. 2018; 327:77-82.
23. Arnalich F, Lopez J, Codoceo R, Jim nez M, Madero R, Montiel C. Relationship of plasma leptin to plasma cytokines and human survivalin sepsis and septic shock. The Journal of infectious diseases. 1999; 180(3):908-11.
24. Zwiech R. Predictive value of conjointly examined IL-1ra, TNF-R I, TNF-R II, and RANTES in patients with primary glomerulonephritis. J Korean Med Sci. 2013; 28(2):261-7.

1. An immunoassay to determine whether a subject has elevated levels of an adiponectin polypeptide and an IL-1Ra polypeptide comprising or consisting of the steps:
i) obtaining a biological sample from a subject to be tested;
ii) forming a preparation comprising said sample and an antibody or antibodies that bind adiponectin and an antibody or antibodies that bind IL-1Ra to form an antibody/adiponectin polypeptide complex and an antibody/IL-1Ra polypeptide complex;
iii) detecting each complex; and
iv) comparing the level of adiponectin and IL-1Ra to a relevant matched control.

2. An immunoassay method to determine whether a subject is suspected of having early stage pancreatic ductal adenocarcinoma or pancreatitis comprising or consisting of the steps:
i) obtaining a biological sample from a subject to be tested;
ii) forming a preparation comprising said sample and an antibody or antibodies that bind an adiponectin and an antibody or antibodies that bind IL-1Ra to form an antibody/adiponectin polypeptide complex and an antibody/IL-1Ra polypeptide complex;
iii) detecting each complex; and
iv) comparing the level of adiponectin polypeptide and IL-1Ra polypeptide to a relevant matched control.

3. (canceled)

4. The method according to claim 1, wherein the adiponectin polypeptide comprises the amino acid sequence set forth in SEQ ID NO: 1, or a polymorphic sequence variant thereof.

5. The method according to claim 1, wherein the level of the adiponectin polypeptide is increased at least 2-fold compared to said normal matched control.

6. The method according to claim 1, wherein the IL-1Ra polypeptide comprises the amino acid sequence set forth in SEQ ID NO: 2, or a polymorphic sequence variant thereof.

7. (canceled)

8. The method according to claim 1, wherein said subject is pre-screened to determine whether the subject is pre-diabetic or has early stage type 2 diabetes.

9. The method according to claim 8, wherein said subject is tested for diabetes mellitus.

10. The method according to claim 1, wherein the level of CA19-9 is determined as a measure of whether the subject has or is predisposed to pancreatic ductal adenocarcinoma (PDAC).

11-12. (canceled)

13. The method according to claim 1, wherein said biological sample is urine, blood, blood plasma or serum.

14. The method according to claim 1, wherein said antibody that binds adiponectin, IL-1Ra and optionally CA19-9 is a monoclonal antibody.

15. The method according to claim 1, wherein the subject is imaged, to determine whether the subject has early stage pancreatic ductal adenocarcinoma or pancreatitis.

16. A treatment regimen for early stage pancreatic ductal adenocarcinoma or pancreatitis comprising:
i) conducting the method according to claim 1 on a subject suspected of having early stage pancreatic ductal adenocarcinoma or pancreatitis; and
ii) treating the subject for early stage pancreatic ductal adenocarcinoma or pancreatitis if the method determines said subject has or is susceptible to early stage pancreatic ductal adenocarcinoma or pancreatitis.

17. The regimen according to claim 16, wherein said treatment is the resection of tumour tissue.

18. The regimen according to claim 16, wherein said treatment is the administration of one or more chemotherapeutic agents.

19. The regimen according to claim 18, wherein said therapeutic agent is selected from the group consisting of: FOLFIRINOX (oxaliplatin, leucovirin, irinotecan and fluorouracil), Gemcitabine, GemCap (gemcitabine and capecitabine), FOLFOX (oxaliplatin, fluorouracil and folinic acid) and Nab-paclitaxel with gemcitabine.

20. A solid support comprising immobilised antibodies that specifically bind adiponectin and/or IL-1Ra.

21. The solid support according to claim 20, wherein said solid support further comprises an immobilised antibody that specifically binds CA19-9.

22. The solid support according to claim 21, wherein said antibodies that specifically bind adiponectin, IL-1Ra and CA19-9 are monoclonal antibodies.

23. A point of care device comprising a solid support according to claim 20.

24. A kit comprising or consisting of:
an antibody that specifically binds an adiponectin polypeptide;
an antibody that specifically binds an IL-1Ra polypeptide; and
secondary antibodies that bind said monoclonal antibody that binds an adiponectin polypeptide and secondary antibodies that bind said monoclonal antibody that binds an IL-1Ra polypeptide wherein said secondary antibodies comprise separate detectable labels.

25. The kit according to claim 24, wherein said kit further comprises an antibody that binds CA19-9 and a secondary antibody that binds CA19-9 comprising a separately detectable label.

26-27. (canceled)


SEQUENCE LISTING (2 sequences)

SEQ ID NO: 1 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 177 aa, Homo Sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE